(19) United States
(12) Patent Application Publication
(10) Pub. No.: US 2009/0130753 A1
(43) Pub. Date: May 21, 2009

(54) Method for Obtaining and Storing Multipotent Stem Cells

(76) Inventors: Russell Bowermaster, Dublin, OH (US); Thomas H. Bob, Powell, OH (US)

Correspondence Address: MAGINOT, MOORE & BECK, LLP;CHASE TOWER, 111 MONUMENT CIRCLE, SUITE 3250, INDIANAPOLIS, IN, 46204, US

(21) Appl. No.: 12/360,397

(22) Filed: Jan. 27, 2009

Related U.S. Application Data
(63) Continuation of application No. 11/417,719, filed on May 4, 2006, now abandoned.

(51) Int. Cl. C12N 5/06; C12N 5/08

(52) U.S. Cl. 435/350; 435/325; 435/366

(57) ABSTRACT

An isolated tissue is provided comprising a source of millions of postnatal stem cells expressing embryonic stem cell markers. A method is also provided for storing or banking such isolated tissue to provide stems cells for later therapeutic use for the donor or for allogenic transplant with donor permission.

### CROSS-REFERENCE TO RELATED APPLICATIONS

This application claims is a continuation of and claims priority to co-pending utility application Ser. No. 11/417,719, filed on May 4, 2006, which claims priority to provisional application 60/668,183, filed on May 15, 2005, the disclosure of which is incorporated herein by reference.

### BACKGROUND

The invention relates to isolated tissues comprising stem cells and to methods of banking stem cells for future use.

Stem cells provide an attractive option for therapies ranging from diabetes treatment, to cancer therapy, bone reconstruction, tooth reconstruction, and reconstructive surgery following accident or disease. The term “stem cell” has been applied to cells at various stages of differentiation, but the most desirable cells are those that are “undifferentiated,” or resemble early embryonic cells which have not yet become committed to a particular differentiation pathway or cell lineage. Studies have shown that these cells express certain surface markers, such as Oct 4 and SSEA1, and do not express certain other markers, such as Sca1. These cells are described as “multipotent” because they can differentiate to form cells from a variety of different tissue types. Multipotent cells generally can differentiate to form at least one cell type of endodermal, ectodermal, or mesodermal origin. “Pluripotent” cells are presumed to be able to differentiate into essentially all cell types. “Embryonic” stem cells, which are derived from in vitro fertilizations or from fetal tissue, are multipotent and often considered to be pluripotent.

Because embryonic stem cells are isolated from human embryos or fetal tissue, there are ethical issues associated with their use. Even if those issues were not considered, however, development of adequate supplies of stem cells of embryonic origin can be difficult and cost prohibitive. Therefore, other sources of stem cells have been investigated.

Non-embryonic stem cells, or stem cells isolated from a source other than a mammalian embryo, have been isolated, and some of these cells have been described as multipotent. Non-embryonic cells have been found, for example, in bone marrow, in cord blood (derived from umbilical cords of infants at birth), and in amniotic fluid (derived from amnion harvested during the first trimester of human pregnancy). Non-embryonic, or postnatal, cells are often referred to in various reports as “adult” stem cells, although some suggest that among the postnatal stem cells there may be differences between cells derived from children and from more developmentally mature adults. The primary difficulties encountered with these cells have been a limited number of cells and difficulty culturing and growing the isolated cells. Two to five milliliters of amniotic fluid, for example, have been reported to contain approximately 1-2×10⁴ live cells per milliliter.

What are needed are readily available sources of stem cells to provide a significant number of multipotent cells that are readily cultured and reproduced to provide cells that can be used when needed to restore damaged tissue and to provide needed diagnostic therapies.

### SUMMARY

The present invention relates to an isolated tissue comprising millions of stem cells. The cells are relatively homogeneous and exhibit markers associated with embryonic stem cells. The isolated tissue also comprises cells that are relatively easy to culture and reproduce.

The invention also relates to a method of storing, or banking, stem cells from a mammalian donor, such as a human donor. In the method, tissue comprising dental papilla associated with a tooth that has not yet erupted to take its place as a permanent tooth in the human dentition is isolated from the oral cavity and placed into an appropriate solution for preserving the tissue. The tissue is associated with information that identifies that tissue as belonging to a specific individual donor, and preserved for later use by that donor. The isolated tissue can therefore be stored for transplant into the original donor or an HLA-matched recipient.

### BRIEF DESCRIPTION OF THE DRAWINGS

FIG. 1 is an illustration of the pattern of permanent teeth in the upper and lower jaw of a human.

FIG. 2 is an x-ray illustrating pre-erupted teeth (indicated by arrows 1) with associated mesenchymal dental papilla (indicated by arrows 2).

FIG. 3 is an illustration of a human tooth with associated dental papilla.

FIG. 4 is a series of illustrations depicting development of a human permanent tooth.

FIG. 5 shows photographs of histological sections illustrating early development of a tooth.

FIG. 6 is an x-ray of an early adolescent, illustrating a second molar with pulp enclosed by enamel 1 and an un-erupted third molar with dental papilla containing mesenchymal cells 2.

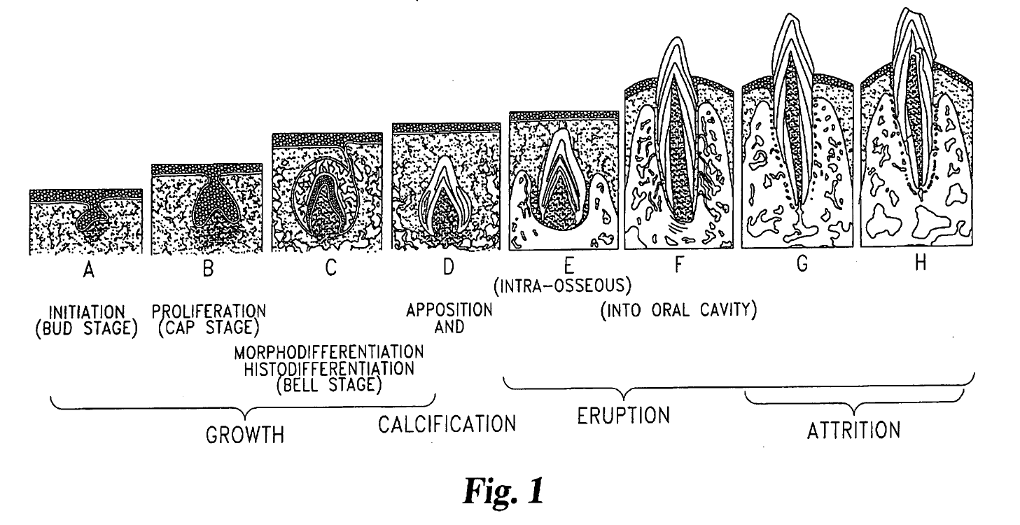

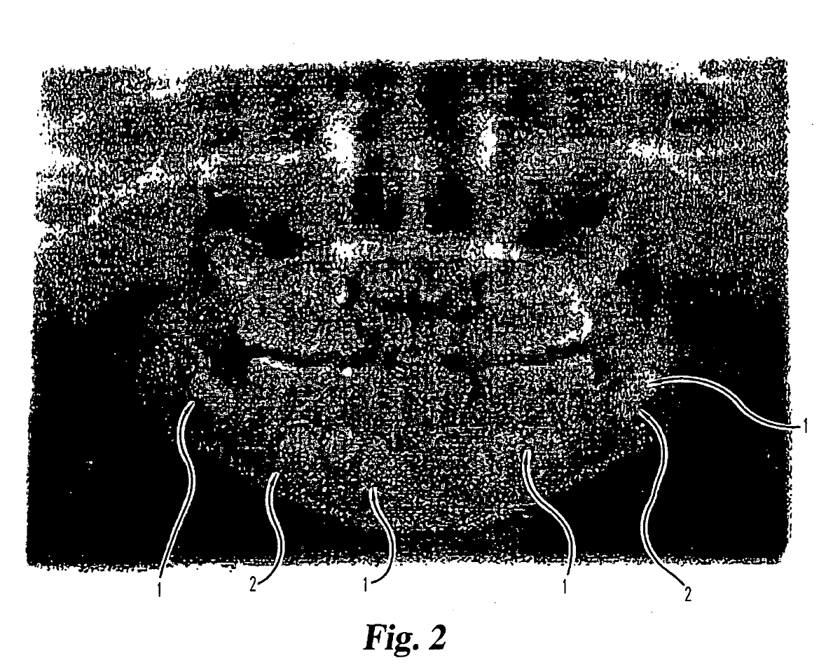

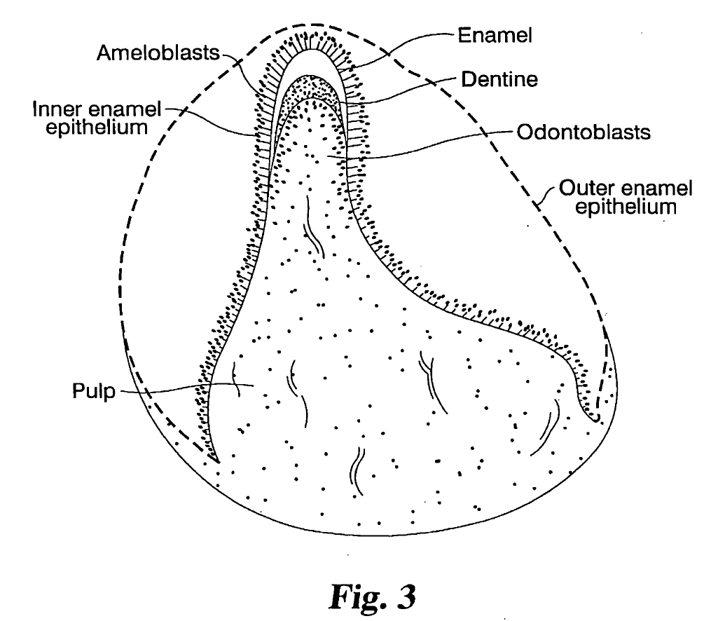

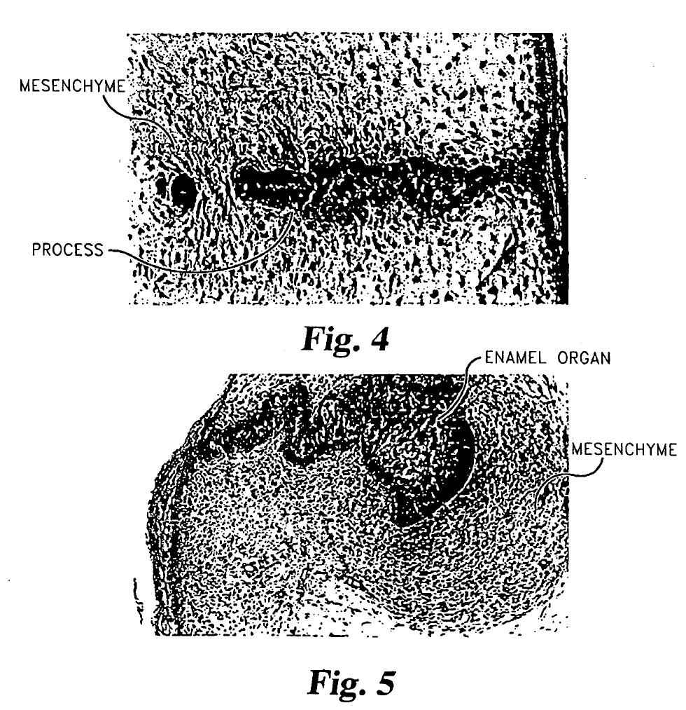

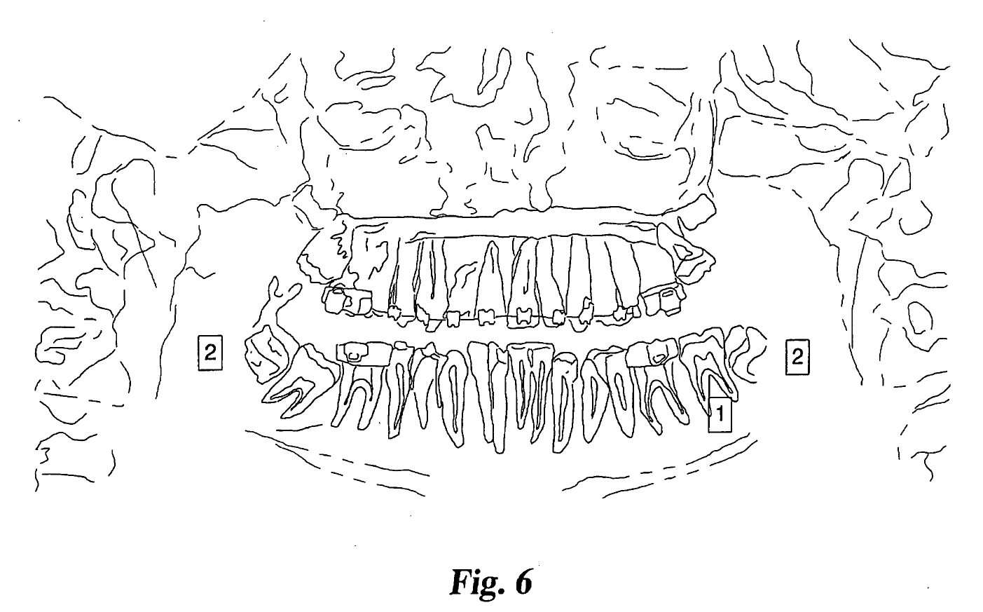

### DETAILED DESCRIPTION

The inventors have discovered a source of millions of stem cells that are relatively homogeneous, grow well, and express markers generally associated with embryonic stem cells (i.e., undifferentiated cells). These cells can be readily obtained by isolating a mesenchymal dental papilla from an un-erupted tooth such as, for example, an un-erupted third molar. As used herein, “isolated” or “isolating” refers to removal of the tissue mass containing stem cells from the oral cavity of a mammal, especially a human. For each such un-erupted tooth, a tissue mass can be isolated to provide up to about 8 to 12 million cells per tooth.

Previous reports, such as that provided in United States patent application publication number 2004/0058442, have indicated that a population of more differentiated dental pulp stem cells exists in the oral cavity, and can be isolated from the periodontal ligament of any human permanent tooth or from a human subject at least about 18 years of age for use in repairing damage to the teeth. A more undifferentiated type of stem cell has been discovered in the dental pulp of an exfoliated deciduous tooth from a six-year old child. These cells were reported to be long-lived and to grow rapidly in culture. Only approximately eight to twelve of these cells were found in each tooth, however.

What the present inventors have unexpectedly discovered is that between the stage of oral development in which the deciduous tooth is found to have a very limited number of undifferentiated cells and the stage in adulthood when the periodontal ligament carries cells that can regenerate the ligament and related tissue lies a development state when tissue isolated from the oral cavity can provide millions of stem cells that express embryonic markers, are relatively easy to culture, and grow rapidly in culture to produce millions more such cells. These cells can be obtained by isolating the developing dental pulp, or dental papilla or any pre-erupted permanent tooth in a mammal.

A tooth bud is a knoblike primordium that develops into an enamel organ surrounded by a dental sac, encasing the dental papilla. Dental papilla is a mass of mesenchymal tissue that ultimately differentiates to form dentin and dental pulp. The dental sac ultimately differentiates to form the periodontal ligament. Tooth buds appear in early childhood, with the last, the third molar, beginning to form at approximately four years of age in a human child. By the time the twenty deciduous teeth have erupted, the first permanent molars are also erupted or erupting, and there are approximately 28 tooth buds for permanent teeth in various states of development in the tissue beneath the deciduous teeth. By the time the teeth erupt, the enamel organ has generally encased the dental pulp. Prior to eruption, however, the mesenchymal tissue may be surgically removed to provide an isolated tissue comprising millions of stem cells, as the inventors have demonstrated.

Any tooth bud or un-erupted tooth may provide an isolated tissue according to the present invention. A particularly attractive source of isolated tissue is the un-erupted third molar, since these developing teeth are often surgically removed because there is insufficient room in the oral cavity for them to erupt or they are not developing normally and may force other teeth out of alignment if they are not removed. Third molars, often called “wisdom teeth”, generally erupt between the ages of 17 and 21. Second molars usually erupt between the ages of 11 and 13, and third molars may be detected by x-ray at about this time. If there is not sufficient room for the third molar or it is not developing normally (e.g., some third molars appear to be growing “sideways” in maxilla or mandible), the molar may be surgically removed at this point so that it cannot become impacted (which may occur if the developed tooth has not reached its appropriate final position by adulthood) or produce misalignment of the other teeth as it develops.

Third molars are customarily removed from pre-teen and teenage patients while the teeth are still developing, and while the primordial bulb still contains millions of stem cells. Since approximately 800,000 third molars (generally the last set of teeth to erupt) are removed each year in the United States alone, and the inventors have demonstrated that each of these teeth comprises an associated tissue mass that contains approximately 8 to 12 million cells per tooth, removal of four third molars from one individual may provide a minimum of approximately 20 million multipotent stem cells. A majority of these cells have been shown to be Oct4 positive, SSEA1 positive, SCAL negative, MART-1 negative, TRA80-1 positive, SSEA-4 negative, CD117 negative and TRA60-1 negative, indicating that the cells are primitive, multipotent stem cells that may be induced to differentiate into a variety of cell and tissue types.

A primordial oral tissue, such as mesenchymal dental papilla of an un-erupted third molar, may be extracted by an oral surgeon using methods known to one skilled in the art, but with care not to generate significant amounts of heat at or immediately surrounding the tooth and associated tissue during extraction. The extracted tooth is associated with tissue that contains fully undifferentiated mesenchyme (dental papilla) from which mesenchymal stem cells can be isolated. Stem cells in this tissue may be identified visually by histologic evaluation and detection of large elongated cell bodies and nuclei, and may be separated by standard cell sorting techniques, such as fluorescence activated cell sorting (FACS) using markers associated with undifferentiated cells. Mesenchymal stem cells can be isolated by trimming the extracted tissue under a binocular microscope at 45× magnification and treating the cells with 1% displace solution (weight/volume) at 15 degree C. for about 1-2 hr. The separated cells can then be washed and treated with 0.1% soybean trypsin inhibitor (weight/volume) for 15 min.

Tooth development begins by formation of the bud, which then becomes the dental “process.” Mesenchyme cells cluster around the base of the process, and the process will eventually become the enamel organ. The enamel organ will eventually enclose the mesenchyme, which will form the dental pulp. While the tooth has not matured sufficiently to enclose the mesenchyme, it is available to be isolated and provides millions of stem cells within a tissue isolate generally no larger than a standard pencil eraser. The tissue is relatively easy to isolate, provides millions of stem cells that are relatively easy to grow and culture, and is available from a vast number of genetically diverse individuals. More of the tissue is available in association with a molar, given the size, shape and pattern of development of the tooth itself. However, a dental papilla associated with any developing un-erupted tooth will produce an isolated tissue of the present invention and a source of millions of stem cells. The permanent teeth in a human usually begin to appear at about age six, but the third molars may not appear until an individual reaches an age of approximately seventeen to approximately twenty-five years. Therefore, the age range within which un-erupted teeth and developing teeth may provide a source of isolated tissue comprising stem cells encompasses childhood, adolescence, and, in some individual, early adulthood.

The discovery of a tissue comprising such a significant number of stem cells in the oral cavity makes possible a method of storing, or “banking”, stem cells literally from a minimum of hundreds of thousands of donors. These cells can be stored for donors so that they may be cultured to differentiation if needed later in life. The cells may also provide a source of a significant genetic variety of stem cells for research purposes. For drug screening, for example, it is desirable to utilize a variety of stem cells from different individuals so that the lines utilized represent genetically diverse populations.

Banking such a variety of cells, particularly in such significant numbers, also provides a method for providing immunologically matched cell and tissue types from allogeneic donors to recipients in need of cells for tissue repair or other therapeutic use.

Generally, for example, an individual patient may provide four mesenchymal bulbs, thereby providing two teeth with associated tissue that can be stored at one storage facility and two that can be stored at a redundancy laboratory. The tissues can be stored by standard cell and tissue preservation methods known to those skilled in the art. Such methods have, for example, been described for cord blood preservation. Methods previously described for cell and tissue preservation include programmed freezing, which provides gradual temperature decrease (usually 1 degree Celsius per minute) until cells are stably frozen without significant damage, and vitrification, which provides rapid freezing that transforms cells and liquids to a more solid state. Cells thus preserved have been stored for periods of years and demonstrated to be viable and capable of being cultured upon reconstitution. Methods and compositions for cell preservation have been described in numerous publications and patents, including, for example, U.S. Pat. No. 6,743,575 (Wiggins, et al.), U.S. Pat. No. 6,653,062 (DePablo, et al.), U.S. Pat. No. 6,632,666 (Baust, et al.), U.S. Pat. No. 5,071,741 (Brockbank, K.), U.S. Pat. No. 5,110,722 (Brockbank, et al.), and U.S. Pat. No. 6,740,484 (Khirabadi, et al.), and Electronic J. of Oncology, 1999, 1, 97-102 (Clapisson, et al.).

In a typical scenario provided by the method of the invention, a patient undergoing third molar excision, for example, would be provided the option of having the excised teeth and associated tissue preserved and shipped for storage at a facility that would store the tissues for a number of years. A kit may be provided to the oral surgeon to provide, for example, appropriate sterile solutions for hydration and transport of the tissues to a facility for preservation, such as cryopreservation. Alternately, the oral surgeon's on-site laboratory facilities may include cryopreservation equipment and methods so that the tissues can be cryopreserved prior to shipping to a storage facility.

An appropriate storage facility for banking stem cells would, generally, comprise a facility having the necessary cryopreservation or other cell preservation equipment and a method for cataloguing samples as they are stored so that the appropriate samples can be matched to the donor when a request is later made for the banked cells. Preferably, of course, such a facility will follow established practices qualifying as Good Laboratory Practices according to the guidelines established by the United States Food and Drug Administration.

In one embodiment, for example, kits could comprise vials tagged with individual RFID tags or UPC labels so that the vials may be tracked from the time the cells are placed in the vials until they are returned to the donor upon request. In one embodiment, a dentist or oral surgeon may be provided with an RFID “reader” so that a tagged vial may be identified while in the office of the dental professional and associated with the necessary donor information by inputting the appropriate data into a computer database associated with the RFID reader in the oral surgeon's or dentist's office. By networking the computer databases such as through internet access to a central database, it would therefore be possible to transmit the necessary data for sample identification to the storage facility so that it could be associated with the sample upon arrival of the sample at the facility and scanning of the vial upon receipt.

The method provided herein preferably provides for one or more redundancy labs in order to protect against the possibility that failure of preservation equipment at one storage facility might destroy the donor tissue and stem cells. As used herein, “lab” or “laboratory” generally refers to a facility where appropriate cell preservation equipment and methods are available, and personnel are trained to utilize such equipment and methods so that cells can be stored using appropriate sterile techniques, monitoring of storage conditions, and tracking of samples so that samples are correctly associated with the appropriate donor throughout collection, transit to the storage facility, storage, and return to the donor upon request.

Among the several significant advantages provided by the method of the present invention is that ability to store significant numbers of autogenetic stem cells so that they will be available to the donor if needed as the donor ages. Due to the sheer numbers of cells available, the method also makes it possible to provide allogeneic matched cells for individuals who are in need of such cells, provided that the donor has given informed consent to use a part of the cells for this purpose. For example, certain patients with hematologic malignancies (i.e., blood or bone-marrow cancers) can be cured with allogeneic stem cell transplantation. In such a procedure, following chemotherapy doctors introduce donor stem cells into the patient's bloodstream, where they migrate to the bone marrow to aid in restoring the immune system. Stem cell donors are usually genetically similar siblings or unrelated volunteers, but over half the patients lack a matched donor. Banking cells from hundreds of thousands, and indeed millions, of donors, many of whom may be willing to share surplus cells with others, provides a significantly larger pool of prospective stem cell donors and increases the opportunities for recipients to be better matched with an appropriate donor.

Stem cells have also demonstrated the ability to repair damaged cardiac muscle tissue, providing hope for individuals with certain cardiomyopathies. Storage of stem cells provides an opportunity for a donor individual to utilize his or her own cells later in life to repair damage to his or her own heart—or to donate a portion of those stored cells to be grown and cultured to repair the heart of another individual through an allogeneic stem cell transplant—the transplant being more likely to provide a tissue “match” because cells might be available from a variety of genetically diverse individuals.

Although the invention has been described in terms of isolation from human tissue and use in human subjects, it is to be understood that isolation and banking of stem cell tissue may also be performed for non-human mammals. Stem cells isolated from primates, for example, may be stored for research purposes or to provide therapeutic benefit for the animal. Such cells might also be used to preserve tissue from species considered to be endangered.

Pet owners, such as dog or cat owners for example, might also desire for their veterinarian to isolate stem cell tissue of their pet and store such tissue for therapeutic use for the donor animal or another animal, to donate a portion of cells for research purposes, or to store cells for reproductive cloning at some future date.

The invention may be further described by means of the following non-limiting example.

### EXAMPLE 1

The following materials were assembled prior to excision of an un-erupted third molar: cold packs, transport media, sterile gloves, sterile drapes and towels, bactericidal oral rinse, bactericidal solution to paint area of extraction, bactericidal and antibiotic media to wash isolated tissue sample, bactericidal irrigation solution and a package for transport of the isolated tissue to the laboratory for preservation.

The surgical procedure was performed under sterile conditions. The un-erupted third molar was carefully removed surgically, taking care not to overheat the tooth structure (such as with a dental appliance) during surgical removal of the tooth and associated tissue. Upon removal of the tooth and adjacent dental papilla tissue, the tooth and tissue were rinsed in bactericidal solution, placed in transport media, and packed in cold packs for transport to the preservation laboratory.

Upon arrival at the laboratory, cells were isolated from the primordial bulb by standard isolation means. Cell counts were performed to determine the number of cells having embryonic stem cell-associated markers. Tissue from mandibular third molars was found to contain more stem cells than tissue from maxillary third molars. An average of approximately 5 million stem cells were isolated from the tissue associated with each tooth. When cultured, approximately five percent (5%) of those cells were established in culture, and grew rapidly to produce approximately 25 million cells.

What is claimed is:

1. A source of multipotent stem cells comprising isolated mesenchymal stem cells all capable of expressing markers associated with embryonic stem cells, the mesenchymal cells isolated from the dental papilla of an un-erupted tooth in a mammal.

2. The source of multipotent stem cells of claim 1, wherein the mammal is a human.

3. The source of multipotent stem cells of claim 1, wherein the mammal is a dog.

4. The source of multipotent stem cells of claim 2, wherein the tooth is a third molar.

5. The source of multipotent stem cells of claim 2, wherein the tooth has not completed its root formation.

6. The source of multipotent stem cells of claim 1, wherein the isolated mesenchymal stem cells are positive for the marker Oct4.

7. The source of multipotent stem cells of claim 1, wherein the isolated mesenchymal stem cells are positive for the markers SSEA1 and TRA80-1 and negative for the markers SCA1, MART-1, SSEA-4, CD117 and TRA60-1.
